(19) United States
(12) Patent Application Publication
(10) Pub. No.: US 2013/0197560 A1
(43) Pub. Date: Aug. 1, 2013

(54) TATTOO ROUND OR SQUARE TWENTY FIVE TO ONE HUNDRED NEEDLES

(71) Applicant: Wade Cheng, South San Francisco, CA (US)

(72) Inventor: Wade Cheng, South San Francisco, CA (US)

(21) Appl. No.: 13/359,353

(22) Filed: Jan. 26, 2012

(51) Int. Cl. A61B 17/34

(52) U.S. Cl. 606/186

(57) ABSTRACT

A tattoo needle made of a wire with a loop at one end and a group of at least twenty eight fine needles arranged in a substantially round configuration at the other end. A tattoo needle made of a wire with a loop at one end and a group of at least twenty five fine needles arranged in a substantially square configuration at the other end.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

1. Field of the Invention

The invention relates generally to tattoo needles and particularly to improved large tattoo needles.

2. Description of the Related Art

During extraction method of tattoo removal, there is significant risk of scarring using traditional tattoo needles (round or magnum needles) to puncture the skin and to cause a chemical formula to come in physical contact with tattoo pigments in the skin (tattoo-over procedure). There are several chemical formulas in the market such as Rejuvi Tattoo Remover, TatgoneInk, EliminInk, Kataderm, salts and acid removal product and so on, which use tattoo-over procedure. The tattoo removal process, using traditional tattoo needles, is also slow. Thus, there is a need for improved tattoo needles that would make such process, and other similar processes, much faster and which would considerably reduce the risk of scarring.

In addition, currently, the micropigmentation or permanent makeup, such as making eyebrows and lip liners, are made by round tattoo needle having 3 or 5 needles. Thus, these are very slow processes. Hence, there is a need for a new and improved tattoo needle that is much faster, so that the labor or time spent can be reduced substantially.

The problems and the associated solutions presented in this section could be or could have been pursued, but they are not necessarily approaches that have been previously conceived or pursued. Therefore, unless otherwise indicated, it should not be assumed that any of the approaches presented in this section qualify as prior art merely by virtue of their presence in this section of the application.

### BRIEF SUMMARY OF THE INVENTION

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

In one exemplary embodiment a tattoo needle is made of a stainless wire with a loop at one end and a group of at least twenty eight stainless fine needles arranged in a substantially round configuration at the other end. In another exemplary embodiment, a tattoo needle is made of a stainless wire with a loop at one end and a group of at least twenty five stainless fine needles arranged in a substantially square configuration at the other end. Thus, the advantages of the new and improved tattoo needles are that they make the tattoo removal process, and other similar processes, much faster and characterized by great reduction or elimination of scarring risk.

The above embodiments and advantages, as well as other embodiments and advantages, will become apparent from the ensuing description and accompanying drawings.

### BRIEF DESCRIPTION OF THE DRAWINGS

For exemplification purposes, and not for limitation purposes, embodiments of the invention are illustrated in the figures of the accompanying drawings, in which:

FIG. 1a illustrates a side view of a round fourteen tattoo needle available as prior art.

FIG. 1b illustrates a close-up view of the end 101 of the tattoo needle depicted in FIG. 1a.

FIG. 2a illustrates a side-perspective view of a traditional tattoo machine available as prior art.

FIG. 2b illustrates a side-perspective view of a coil tattoo machine available as prior art.

FIG. 3a illustrates a side diagrammatic view of a tattoo needle, according to several embodiments.

FIG. 3b illustrates a bottom diagrammatic view of a round tattoo needle having twenty eight fine needles, according to one embodiment.

FIG. 3c illustrates a bottom diagrammatic view of a round tattoo needle having fifty two fine needles, according to another embodiment.

FIG. 3d illustrates a bottom diagrammatic view of a round tattoo needle having one hundred fine needles, according to another embodiment.

FIG. 4a shows examples of actual round tattoo needle having fifty two fine needles.

FIG. 4b shows another example of an actual round tattoo needle having fifty two fine needles and an example of a tattoo needle sleeve.

FIG. 5 illustrates bottom diagrammatic views of exemplary square tattoo needles, according to other embodiments.

### DETAILED DESCRIPTION OF THE PREFERRED EMBODIMENTS

What follows is a detailed description of the preferred embodiments of the invention in which the invention may be practiced. Reference will be made to the attached drawings, and the information included in the drawings is part of this detailed description. The specific preferred embodiments of the invention, which will be described herein, are presented for exemplification purposes, and not for limitation purposes. It should be understood that structural and/or logical modifications could be made by someone of ordinary skills in the art without departing from the scope of the invention. Therefore, the scope of the invention is defined by the accompanying claims and their equivalents.

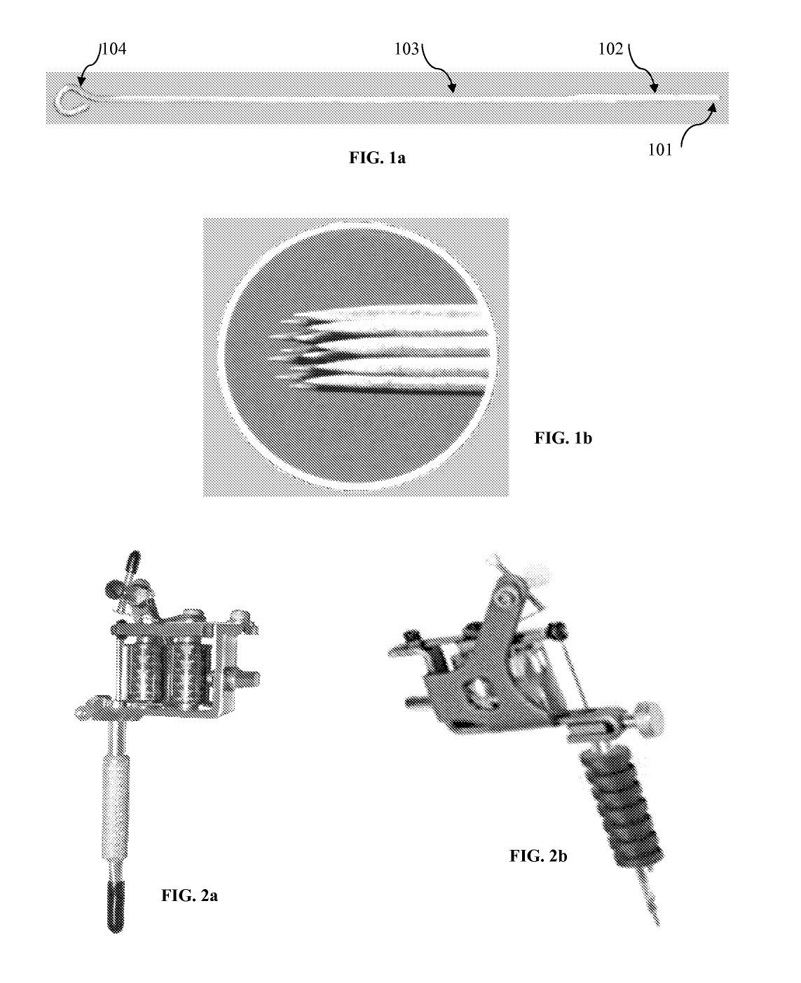

Referring to FIG. 1a, a side view of a round fourteen tattoo needle available on the market is shown. Regular tattoo needle, as the one shown in FIG. 1a, consists of a group 102 of fine solid stainless steel needles which are welded or soldered to one end of a stainless steel wire 103. The wire 103 has a diameter of approximately 1.5 mm (i.e., 0.059 inches) and a length of approximately 139.7 to 146.05 mm (i.e., 5.5 to 5.75 inches). At the other end, opposite to the group of needles 102, the wire 103 is forming a loop 104.

The fine stainless solid needles have a diameter of approximately 02-0.3 mm (i.e., 0.007874-0.011811 inches) and they have a very sharp point at one end (see FIG. 1b). A group of needles may consist of 1, 3, 5, 7 or more in a round bundle (called round needle; see FIG. 1b), or in a straight line (called flat needle; not shown) or in a double line (called magnum needle; not shown). A group of fourteen fine needles of a tattoo needle available on the market is shown in FIG. 1b. The largest round needle available on the market has a group of eighteen fine needles.

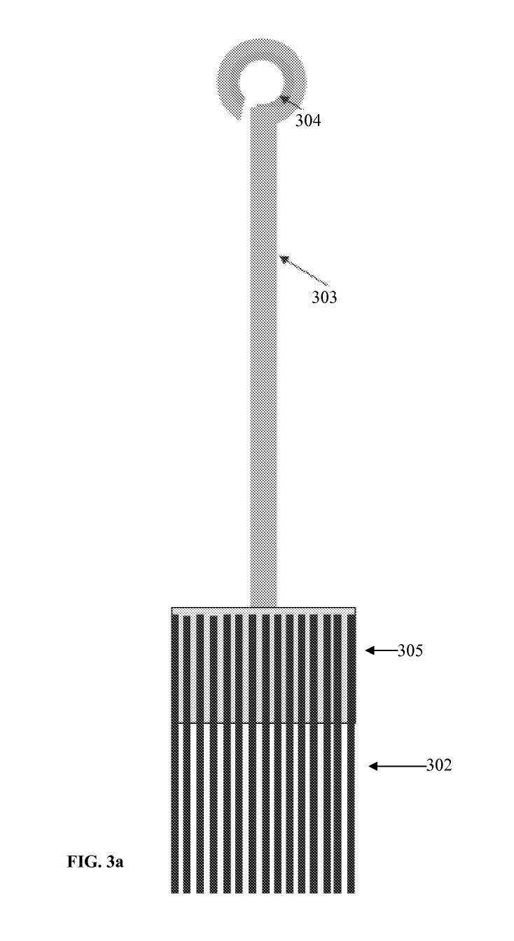

For tattooing purposes, a tattoo needle can be mounted in a traditional tattoo machine, such as the one shown in FIG. 2a, or in a coil tattoo machine, such as the one shown in FIG. 2b.

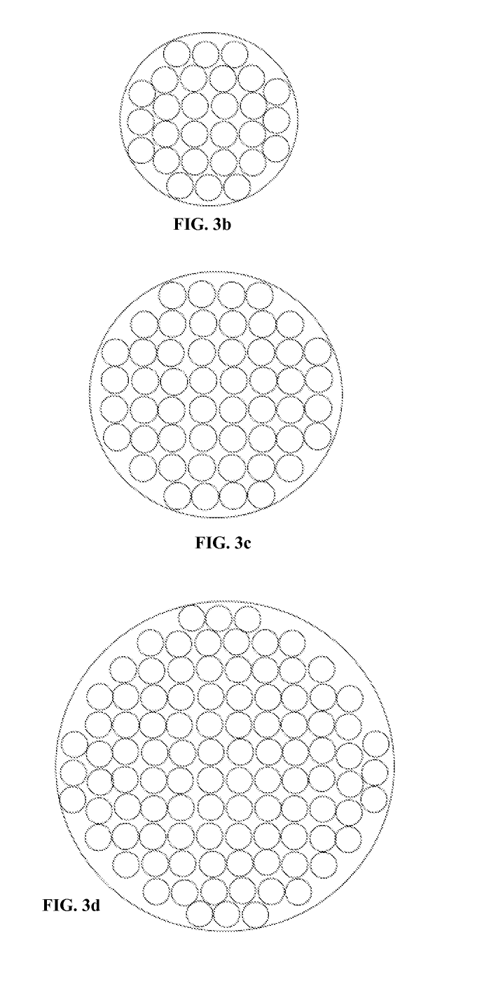

Referring now to FIG. 3a, a side diagrammatic view of a tattoo needle, according to several embodiments, is shown. The shown tattoo needle is made of high grade (304 or 316) stainless steel and has a length of approximately 142 mm (i.e., 5.59 inches). However, other lengths may be used without departing from the scope and essence of the invention. The internal diameter of loop 304 measures approximately 5 mm (i.e., 0.19685 inches). The diameter of the stainless wire 303 measures approximately 1.5 min (i.e., 0.059 inches). Again, these dimensions may be varied, without departing from the scope and essence of the invention.

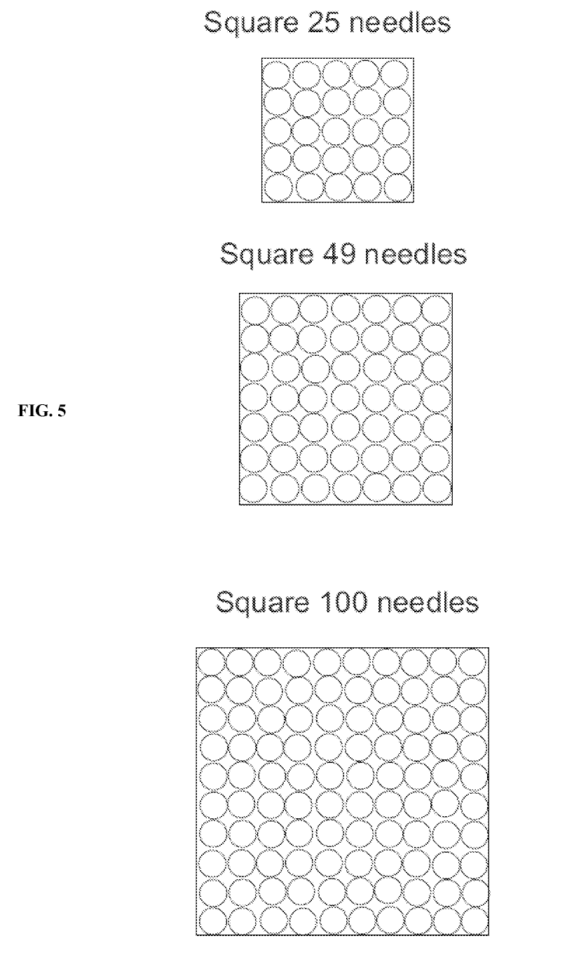

A group 302 of twenty five (square; see FIG. 5), twenty eight (round), or up to one hundred, or even more, stainless fine needles, may be welded or soldered to section 305 (over enlarged for illustration purposes) of the needle, as needed to suit several special applications. Examples of round groups of twenty eight, fifty two and one hundred fine needles we shown in FIGS. 3b, 3c and 3d, respectively, according to embodiments of the invention. However, it should be understood that a group 302 having a different number of tine needles, greater than twenty eight (round) or than twenty five (square), may be used, which may be capable of achieving the same results as described herein, and which, thus, may be substituted for the specific embodiments illustrated and described herein without departing from the scope of the invention.

As shown in FIGS. 3b, 3c and 3d, respectively, the groups of twenty eight, fifty two and one hundred fine needles can be arranged in a substantially round configuration, Thus, this is an advantage of selecting these exact numbers. Furthermore, practice suggests that the number of fine needles needed in the group 302 for obtaining a considerable amount of the beneficial results described herein has to be somewhere in the mid twenties. Thus, the minimum number of fine needles for a square group is twenty five and for a round group is twenty eight.

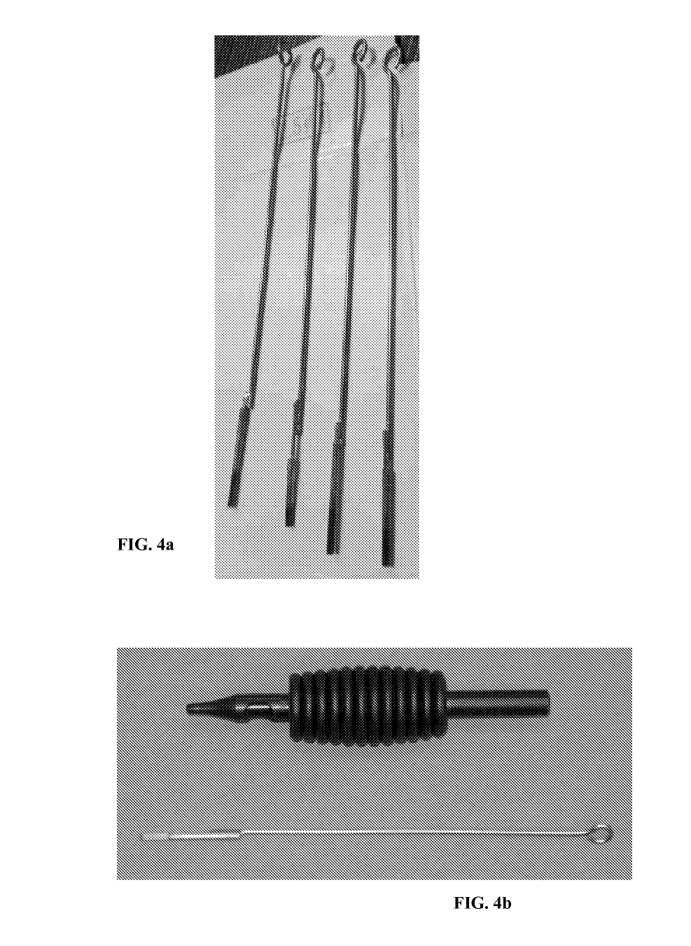

Again, FIG. 4a shows examples of actual round tattoo needle having fifty two fine needles, according to one embodiment, and FIG. 4b shows another example of a round tattoo needle having fifty two fine needles, according to one embodiment, as well as an example of a tattoo needle sleeve, which may be used in conjunction with the round tattoo needle shown.

The invented large round needles (28 fine needles or more) disclosed herein are very good for tattoo removal via chemical extraction method, i.e., delivery of a chemical formulation for contact with tattoo pigments in the skin. The chemical formulation then binds with the tattoo pigments and separates them from the skin. This application is very similar to tattoo-over process in which a chemical formula (a paste, solution or cream) is delivered into unwanted tattoo area using traditional tattoo machines. The invented big round needles (28 fine needles or more) have the advantage of considerably speeding up the chemical formulation delivery process. In addition, and more importantly, the invented big round needles minimize the risk of scarring and other possible side effects such as skin infections. These are very crucial benefits.

The scarring is prevented because the same amount of force used in conjunction with a traditional needle, having for example 14 fine needles, is now distributed to, for example, 52 fine needles. Thus, the pressure on each individual fine, needle is smaller. Furthermore, scarring is also prevented because the improved needle (28 fine needles or more), allows a user to complete a job by going over the same area usually only once or twice, as opposed to using a traditional tattoo needle when repeated action is usually needed over the same area,

The invented large round needles (28 fine needles or more) may also be advantageously utilized for making special tattoo patterns, such as thick frames or contours, considerably faster than with traditional round needles (i.e., round tattoo needle with 18 fine needles or less). They can speed up tattooing process so as to save time and/or reduce labor cost significantly. Again, this is because the improved needle (28 fine needles or more), allows a user to complete a job by going over the same area usually only once or twice, as opposed to using a traditional tattoo needle when repeated action is usually needed over the same area.

The invented large round needles (28 fine needles or more) may also be advantageously used for micropigmentation or permanent makeup such as making eyebrows and lip liners. When the invented large round needles are used, the process is much faster than the traditional micropigmentation or permanent makeup processes. This means that the process is very cost effective and it may also increase customer satisfaction, because of less time for the customer to undergo the respective procedure. It should be understood that in this case, when the invented large round needles are used, a traditional tattoo machine instead of cosmetic tattoo machine or a micropigmentation machine should be used.

FIG. 5 illustrates bottom diagrammatic views of exemplary square tattoo needles, according to other embodiments. Again, practice suggests that the number of fine needles needed in the group 302 (see FIG. 3a) for obtaining a considerable amount of the beneficial results described herein has to be somewhere in the mid twenties. Thus, the minimum number of fine needles for a square group is twenty five, as shown in FIG. 5. A square group of forty nine fine needles and a group of one hundred needles are also shown in FIG. 5, as examples only. It should be understood that other square configurations, such as six by six for a total of thirty six fine needles, may be used.

Tattoo needles having square groups of fine needles may be slightly more difficult to manufacture than the tattoo needles having round groups of fine needles. However, they may have additional usefulness for certain special applications in addition to the scarring prevention and efficiency benefits of the round groups described earlier herein.

It may be advantageous to set forth definitions of certain words and phrases used in this patent document. The term “couple” and its derivatives refer to any direct or indirect communication between two or more elements, whether or not those elements are in physical contact with one another. The terms “include” and “comprise,” as well as derivatives thereof, mean inclusion without limitation. The term “or” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like.

Although specific embodiments have been illustrated and described herein for the purpose of disclosing the preferred embodiments, someone of ordinary skills in the art will easily detect alternate embodiments and/or equivalent variations, which may be capable of achieving the same results, and which may be substituted for the specific embodiments illustrated and described herein without departing from the scope of the invention. Therefore, the scope of this application is intended to cover alternate embodiments and/or equivalent variations of the specific embodiments illustrated and/or described herein. Hence, the scope of the invention is defined by the accompanying claims and their equivalents. Furthermore, each and every claim is incorporated as further disclosure into the specification and the claims are embodiment(s) of the invention.

What is claimed is:

1. A tattoo needle comprising a wire and, associated with one end of the wire, a group of at least twenty eight fine needles arranged in a substantially round configuration.

2. The tattoo needle of claim 1, wherein said wire has a loop at the other end.

3. The tattoo needle of claim 1, wherein said wire is substantially 1.5 millimeters in diameter.

4. The tattoo needle of claim 1, wherein each of the fine needles are substantially 0.2 millimeters in diameter.

5. The tattoo needle of claim 1, wherein said group has fifty two fine needles.

6. The tattoo needle of claim 1, wherein said group has one hundred fine needles.

7. A tattoo needle comprising a wire and, associated with one end of the wire, a group of at least twenty five fine needles arranged in a substantially square configuration.

8. The tattoo needle of claim 7, wherein said wire has a loop at the other end.

9. The tattoo needle of claim 7, wherein said wire is substantially 1.5 millimeters in diameter.

10. The tattoo needle of claim 7, wherein each of the fine needles are substantially 0.2 millimeters in diameter.

11. The tattoo needle of claim 7, wherein said group has forty nine fine needles.

12. The tattoo needle of claim 7, wherein said group has one hundred fine needles.

13. A method of tattoo removal by chemical extraction using a tattoo needle having at least twenty five fine needles.
